(19) United States
(12) Patent Application Publication
(10) Pub. No.: US 2008/0219959 A1
(43) Pub. Date: Sep. 11, 2008

(54) Support Having Nanostructured Titanium Dioxide Film And Uses Thereof

(76) Inventors: Roberta Carbone, Opera (MI) (IT); Pier Giuseppe Pelicci, Opera (MI) (IT); Paolo Milani, Pavia (PV) (IT); Paolo Piseri, Milano (MI) (IT); Emanuele Barborini, Pizzighettone (CR) (IT); Gero Antonio Bongiorno, Cesano Boscone (MI) (IT)

Correspondence Address: ST. ONGE STEWARD JOHNSTON & REENS, LLC, 986 BEDFORD STREET, STAMFORD, CT, 06905-5619, US

(21) Appl. No.: 12/016,716

(22) Filed: Jan. 18, 2008

Related U.S. Application Data
(63) Continuation of application No. PCT/EP2006/064377, filed on Jul. 18, 2006.

(30) Foreign Application Priority Data
Jul. 21, 2005 (EP) 05015869.0

(51) Int. Cl. A61K 35/12; C12N 5/06; A61P 43/00; C12N 15/87

(52) U.S. Cl. 424/93.21; 435/402; 435/456

(57) ABSTRACT

The present invention relates to supports for bioassays and the use thereof in cell culturing and in cell-based methods and assays. More precisely, the invention provides solid materials coated with films of nanostructured titanium dioxide suitable for the immobilisation of viruses and for cell-adhesion. The nanostructured TiO<SUB>2</SUB> film-coated support of the invention is particularly useful for the preparation of microarrays for genetic and phenotypic analysis.

### CROSS-REFERENCE TO RELATED APPLICATIONS

The present application is a continuation of pending International patent application PCT/EP2006/064377 filed on Jul. 18, 2006 which designates the United States and claims priority from European patent application 05015869.0 filed on Jul. 21, 2005, the content of which is incorporated herein by reference.

### FIELD OF THE INVENTION

The present invention relates to supports for bioassays and the use thereof in cell culturing and in cell-based methods and assays. More precisely, the invention provides solid materials coated with films of nanostructured titanium dioxide suitable for the immobilisation of viruses and for cell-adhesion. The nanostructured TiO2 film-coated support of the invention is particularly useful for the preparation of microarrays for genetic and phenotypic analysis.

### BACKGROUND OF THE INVENTION

Since the genome has been completely sequenced the need of exploring the full repertoire of proteins for their function in the normal and pathological conditions has become a main goal for new drug target identification and gene therapy applications [1].

The high throughput analysis for gene function studies requires high efficiency of gene transduction and the possibility to analyze different cellular model systems, in a convenient format, on a suitable support, possibly a slide, where thousands of genes can be analyzed simultaneously by simple methods like immunofluorescence.

Several methods of gene transduction have been proposed including uptake of plasmid DNA by transfection [2], electroporation [3], microinjection [4], and viral infection [5].

The most efficient among these technologies is the virus-mediated gene delivery since different kinds of cellular systems, primary and cancer cells of mammalian origin, have shown to be successfully transduced with different viral vectors.

Titanium dioxide (TiO₂) is known as a biocompatible material [6] and it is widely used in implants. Protein and cell attachment mechanisms on TiO₂ films have been studied [7, 8]. The adsorption of proteins on nanocrystalline TiO₂ films has been studied in [19]. The modification of the surface at the nanoscale has been recognized as important to favour cell adhesion, however the mechanisms influencing the cell attachment on a nanostructured substrate are largely unknown [9].

TiO₂-oligonucleotide nanocomposites have recently been proposed as vectors for the introduction into cells of genetic material [1 O]. These nanocomposites retain the bioactivity of the oligonucleotide DNA and they can be photoactivated to induce nucleic acid endonuclease in view of gene therapy.

The realization of a viral array, where each cluster of cells will be infected by substrate-immobilized viral particles is still a challenge: the method should allow a) viral immobilization on the substrate while maintaining virus activity toward target cells b) the virus should be immobilized but be able to enter target cells c) the substrate of immobilization should be biocompatible to permit cell attachment, infection and proliferation and also be optically transparent.

The technical problem underlying the present invention is therefore to provide novel supports for use in bioassays using virus and/or cells.

The solution to the above technical problem is provided by the embodiments of the present invention as characterized in the claims.

### SUMMARY OF THE INVENTION

In particular, it has been found that nanostructured TiO₂ obtained by deposition of nanoparticles from the gas phase provides a valuable substrate for virus adsorption and cell-adhesion, entirely compatible with cell culture and growth.

According to a first aspect, the invention is directed to a solid support especially suitable for in vitro bioassays, consisting of a biocompatible substrate material coated at least partially with a nanostructured TiO₂ film having viruses and/or cells immobilised on the surface thereof.

Any material suitable for cell or tissue culturing and for cell-based assays can be used as a substrate for TiO₂ film coating, preferably glass, plastic, ceramic, metal or a biodegradable or undegradable biopolymeric materials. As conventionally used, the term “biocompatible” indicates that the material should not affect or interfere with normal cell activities, e.g. in vitro growth and proliferation, nor interact with, or alter, the substances used in the preparation of cell cultures.

The support material may be differently shaped depending on the application sought and on the assay format. Suitable supports include, but are not limited to, slides, e.g. microscope slides, dishes, flasks or plates (such as microtiter plates having multiple, e.g. 96, wells), especially for cell culture and for microarrays for high-throughput techniques. Other suitable forms for the support of the present invention are coverslips, fibers, foams, particles, membranes, porous scaffolds, meshes or implants.

The nanostructured TiO₂ (ns-TiO₂) film according to the invention preferably consists of TiO₂ nanoparticles (i.e. crystallites) with a diameter below 20 nm, embedded in an amorphous matrix (i.e. of TiO₂) with a density below 75% of bulk TiO₂ density. The ns-TiO₂ film can be formed on the substrate material by deposition of nanoparticles from the gas phase onto the substrate, preferably by means of supersonic cluster beam deposition (SCBD) using the apparatus disclosed in U.S. Pat. No. 6,392,188.

Thus, the present invention further relates to a method for the production of the solid support as defined above, which comprises the steps of:

(a) formation of a nanostructured TiO₂ film at least on areas of the substrate material coming into contact with biological material, i.e. virus and/or cells, by deposition of nanoparticles from the gas-phase onto the substrate, for instance by means of supersonic cluster beam deposition (SCBD) using a pulsed microplasma cluster source; and

(b) contacting the surface of the nanostructured TiO₂ film with viruses and/or cells.

Briefly, the SCBD technique consists in the assembling of clusters produced in supersonic expansions. The clusters are aerodynamically accelerated to hyperthermal energies in order to provide an impact energy high enough to create links between the cluster and the growing material, but not such to destroy the structure of the impinging particle. The production process utilizes a cluster source known as Pulsed Microplasma Cluster Source (PMCS). The process allows the deposition of nanostructured thin films with a precise control on cluster mass distribution and kinetic energy. The PMCS technology consists in the generation of clusters by condensation of plasma of the desired material (i.e. TiO₂) with an inert carrier gas. The process can be carried out with the substrate kept at room temperature. Further details of the deposition apparatus and process are provided in U.S. Pat. No. 6,392,188, which is herein incorporated by reference.

The ns-TiO₂ film deposition process can be set to produce either completely or partially coated substrate materials; generally, the ns-TiO₂ film is deposited on the support surfaces which come into contact with the biological material, i.e. viruses and/or cells.

The term “immobilisation” as used herein means that the viruses and/or cells are attached to the surface of the nanostructured TiO₂ film by any chemical (e.g. by using binding partners such as streptavidin/biotin, antigen/antibody etc.) or physical means (e.g. adhesion or adsorption).

Thus, in a further aspect, the invention provides the use of a nanostructured TiO₂ film, preferably obtained by means of supersonic cluster beam deposition using a pulsed microplasma cluster source, as a substrate for virus adsorption or cell adhesion.

An ultraviolet photoelectron spectroscopy (UPS) analysis shows that the valence band of as deposited ns-TiO₂ films is characterized by states with energies between 3 and 9 eV with respect to the Fermi level (E_{F}). In this range, the peak at about 6 eV and the peak at 8 eV correspond to π (nonbonding) and σ (bonding) O 2p orbital. A considerable presence of gap states at 0.8 eV below the E_{F} is observed. These states are related to Ti³⁺ point defects due to oxygen vacancies. The large porosity and the presence of chemisorption sites in ns-TiO₂ films suggest that the attachment of proteins, the adsorption of viruses and the adhesion of cells may be favoured by the presence of positive electric charge distributed on the surface and by the large active surface area.

Cells or viruses can be adhered to or adsorbed on the surface of TiO₂ films by simple contact of cell preparations (e.g. suspensions) or virus-containing solutions.

According to an alternative embodiment of the invention, streptavidin, avidin or neutravidin is immobilized on the ns-TiO₂ film deposited on a suitable support in order to interact with biotinylated viruses for their attachment on the ns-TiO₂ film.

The present invention further relates to the use of the solid supports according to the invention for infection of cells with viruses, in particular for virus-mediated gene delivery to cells.

Therefore, the present invention generally provides a method for cell infection with viruses in vitro comprising the steps of:

(a) providing a solid support of a biocompatible substrate material being at least partially coated with a nanostructured TiO₂ film; and

(b) culturing cells on the nanostructured TiO₂ film-coated support in the presence of an infecting virus.

The method for cell infection according to the invention can thus be carried out by simply culturing the cells on the nanostructured TiO₂ film-coated support in the presence of an infecting virus, i.e. without utilizing a binding pair such as biotin/streptavidin. Thanks to the peculiar characteristics of ns-TiO₂ films, in fact, viruses adhere to the substrate surface and infect the cells as efficiently as with infection enhancers such as polybrene or other polycations; unlike polybrene and polycations, however, ns-TiO₂ coated supports do not cause toxicity problems nor affect cell functionality [11].

According to a preferred embodiment, the infection method comprises the steps of:

(a) providing a solid support of a biocompatible substrate material being at least partially coated with a nanostructured TiO₂ film;

(b) contacting the nanostructured TiO₂ film-coated support with viruses;

(c) contacting the virus adhered on the surface of the nanostructured TiO₂ film-coated support with a cell preparation; and

(d) culturing the cells for a time sufficient for the infection to occur.

Alternatively, the infection method comprises the steps of:

(a) providing a solid support of a biocompatible substrate material being at least partially coated with a nanostructured TiO₂ film;

(b) immobilising streptavidin, avidin or neutravidin on the nanostructured TiO₂ film coating;

(c) contacting the nanostructured TiO₂ film-coated support with a biotinylated virus so as to form a complex of biotinylated virus with the immobilised streptavidin, avidin or neutravidin;

(d) contacting the complex with a cell preparation; and

(e) culturing the cells for a time sufficient for the infection to occur.

The production of biotinylated viruses, e.g. retroviruses can be carried out according to methods known in the art, e.g. as described in [18].

Alternatively, the infection method of the invention comprises the steps of:

(a) providing a solid support of a biocompatible substrate material being at least partially coated with a nanostructured TiO₂ film;

(b) adding a cell preparation to the nanostructured TiO₂ film-coated support;

(c) culturing the cells for an appropriate period of time;

(d) adding a viral supernatant; and

(e) culturing the cells for a time sufficient for the infection to occur.

Preferred viruses for use in the present invention are retroviruses, adenoviruses, adeno-associated viruses (AAV) and any other viruses that can be utilized as vectors for genetic manipulation of cells. “Cells” according to the present invention comprise prokaryotic cells such as bacteria as well as eukaryotic cells such as yeast, plant cells, animal cells, preferably mammalian cells, especially human cells.

In general, any methodology suitable for virus-mediated gene delivery to cells can be carried out using ns-TiO₂ film-coated supports according to the invention. Virus immobilisation on ns-TiO₂ films can be obtained, for example, by means of an anchor molecule such as retronectin, a chimeric peptide of human fibronectin which, when coated on the surface of a suitable support (e.g. petri dishes or flasks), significantly enhances retrovirus-mediated gene transduction into cells [12]. Alternatively, viruses or cells can be genetically modified so as to expose on their surfaces an antigen or binding peptide which is recognized and bound by an antibody or protein immobilised on the ns-TiO₂ film.

In a preferred embodiment, ns-TiO₂ film-coated supports according to the invention are used to set up microarray systems. Therefore, the present invention also provides a microarray device which comprises a solid support of the invention wherein the nanostructured TiO₂ film is in the form of a micro- or nano-pattern. The extremely high collimation obtainable with the SCBD technique allows in fact the production of micro and nano-patterned ns-TiO₂ films with a very high resolution [13, 14]. The micro- or nano-patterned films can be differentially functionalised depending on the desired application. For example, supports coated with microarray-patterned ns-TiO₂ films can be used in genetic and phenotypic assays. For these applications, viruses carrying different genetic inserts are spotted on the microarray and used to infect cells. Infected cells are then analysed for the integration or expression of the exogenous genetic material using suitable detection systems.

In addition, the ns-TiO₂ coated materials according to the invention can be used to develop methods for gene therapy and cell replacement therapy, e.g. systems to perform localized infection through TiO₂ nanoparticles loaded with viruses that can be implanted in specific tissues to favour high levels of local gene transduction. The ns-TiO₂ coated materials according to the invention are also useful for ex vivo gene therapy. A typical method for ex vivo gene therapy according to the invention comprises the steps of recovering cells to be genetically modified from a patient, establishing a primary cell culture, infecting the cells by the infection method of the invention with a virus that carries the corresponding genetic information, and re-administering the infected cells to the patient.

Therefore, the present invention provides implantable particles or devices, i.e. any two or three dimensional body (e.g. a chip) of a nanostructured TiO₂ film-coated biocompatible material loaded with viruses. The viruses may be adhered to or adsorbed on the surface of the TiO₂ film-coated material, or may be attached thereto by use of a binding pair or other means as described above.

Furthermore, the present invention also relates to a method for gene therapy in vivo comprising the steps of:

(a) providing particles or device of a nanostructured TiO₂ film-coated biocompatible material;

(b) loading the particles or device with viruses; and

(c) implanting the virus-loaded particles into a tissue of a patient.

As mentioned above, the ns-TiO₂ coated materials according to the invention are useful in cell replacement therapy. Thus, a further embodiment of the present invention relates to a cell replacement therapy method comprising the steps of:

(a) providing particles or a device of a nanostructured TiO₂ film-coated biocompatible material;

(b) loading the particles or device with cells to be replaced in a patient, preferably genetically modified cells; and

(c) implanting the cell-loaded particles or device into a tissue of a patient.

### BRIEF DESCRIPTION OF THE DRAWINGS

The invention is further illustrated by the following non-limiting Examples and by the attached Figures.

FIG. 1: Streptavidin adsorption on ns-TiO₂. Spotting of streptavidin-Cy3 on a layer of ns-TiO₂. Incubation at 37° C. in culture medium for different time periods (O, 8, 24 and 48 h).

FIG. 2A: Classical infection of melanocytes with retroviral supernatant (GFP).

FIG. 2B: ‘Reverse infection’ of melanocytes with retroviral supernatant (GFP).

Comparison of different substrates (plastic, gelatin coated coverslips and ns-TiO₂ in relation to infection efficiency

FIG. 3: Retroviral microarray with U2OS cells.

### DETAILED DESCRIPTION OF THE INVENTION

### Examples

1. Preparation of ns-TiO₂ Substrate

Nanostructured TiO₂ films have been deposited by a Supersonic Cluster Beam Deposition (SCBD) apparatus equipped with a Pulsed Microplasma Cluster Source (PMCS) [15]. Briefly, a titanium target is sputtered by a confined plasma jet of an inert gas (He or Ar). Sputtered Ti atoms thermalize within the inert gas and condense to form clusters. The Ti clusters are either oxidized by interaction with residual gas in the background vacuum or by the introduction of a suitable amount of oxygen in the process. The mixture of clusters and inert gas is then extracted in vacuum through a nozzle to form a seeded supersonic beam which is collected on a substrate located in the beam trajectory. The kinetic energy of the clusters is low enough to avoid fragmentation and hence a nanostructured film is grown. The mass distribution of the clusters can be controlled by aerodynamic focusing in order to tailor the nanostructure of the film [16].

2. Cell-Infection Assay on ns-TiO₂ Array

Viral vectors are prepared by Ca(PO₄)₂ transfection procedures in Amphotropic Phoenix packaging cells [17]. Cells are biotinylated in vivo [18] and viral supernatant is collected, concentrated 10 times with 8% PEG8000 after overnight incubation at 4° C. and aliquoted in presence of 100 μg/ml of a stabilizing sugar, preferably trehalose, at −80° C.

Viral titration indicates different viral concentration ranging from 108 to 10¹² cfu/ml.

A monolayer of protein (streptavidin) ranging between 1 μg/ml to 0.1 μg/ml in Hepes 10 mlWNaCl 150 mM buffer is prepared on the nanostructured TiO₂ slide by robotic spotting, incubated to allow adsorption, and the monolayer is stabilized by a treatment with 10% serum. Biotinylated virus is then deposited by robotic spotting on the functionalized substrate: after an incubation time to allow virus binding, wash steps eliminate the viral excess and cells are plated on the substrate.

To perform analysis of the infected array after 48-72 hours cells are processed for immunodetection by microscopy or treated with the appropriate antibiotic (puromicine, hygromicine, G418) to perform selection and obtain a homogeneous population of cells growing in clusters, expressing or down-regulating at high efficiency the gene of interest. At the end of selection the slide is processed for microscopy or scanner detection.

3. Streptavidin Adsorption on ns-TiO₂

0.1 μg/ml streptavidin labelled with Cy3 in 150 mM NaCl, 10 mM Hepes was spotted on a slide coated with nanostructured TiO₂ film.

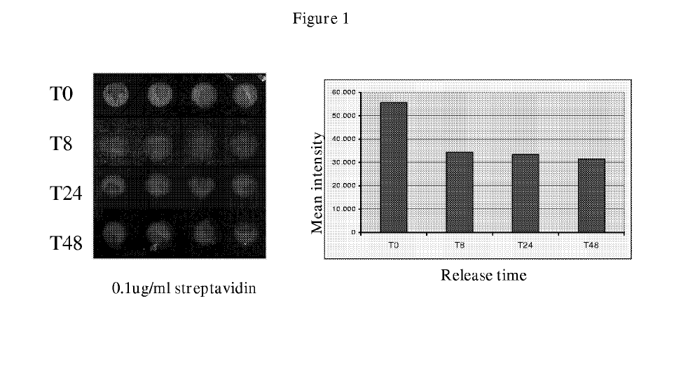

The slide was incubated in saline medium at 37° C. for different time points (0, 8, 24, 48 hr) to verify whether the spotted protein had been stably adsorbed onto the TiO₂ surface. Afterwards, the slide was scanned to determine the fluorescence intensity. The results (FIG. 1) show that after 8 hrs the fluorescence intensity is constant, indicating that streptavidin molecules form a stable layer adsorbed on TiO₂.

4. ns-TiO₂-Mediated Melanocyte Infection in the Absence of Polybrene

Primary melanocytes were used as target cells. Briefly, for the classical infection protocols cells were plated on a plastic support (control) and on ns-TiO₂ coated coverslips. After 24 hours, the cells were infected for 12 hrs with a GFP-expressing virus in solution in the presence or absence of polybrene. 72 hrs later, the cells were fixed with 4% paraformaldehyde for 10 minutes and the nuclei were stained with DAPI.

Cells were analysed with a fluorescence microscope. The infection efficiency and the mean fluorescence intensity were calculated for each sample using an image analysis software.

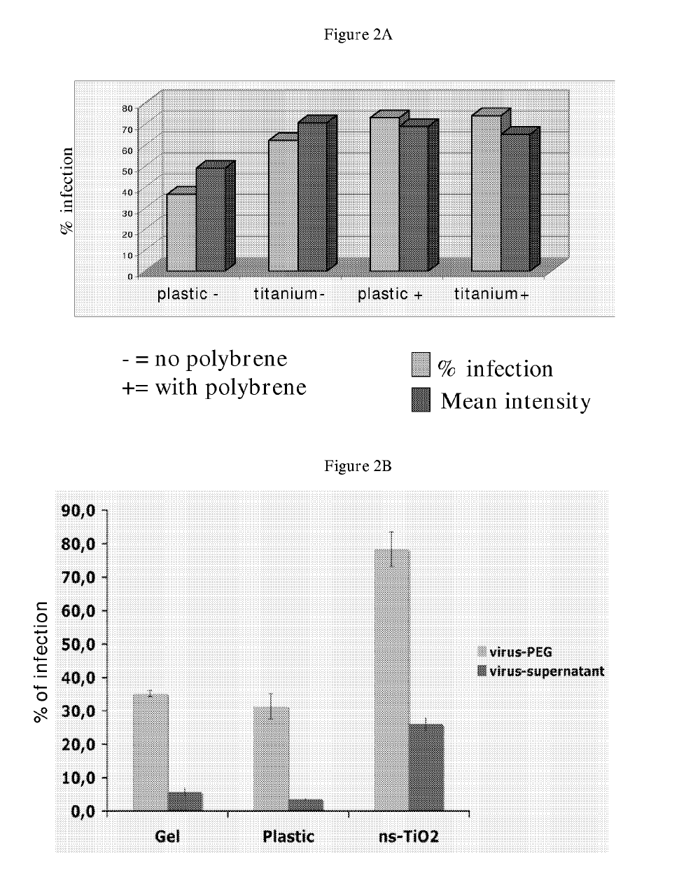

The results (FIG. 2A) show that the infections via ns-TiO₂ in the absence of polybrene and, on the plastic support in the presence of polybrene, respectively, have the same efficiency and mean intensity.

For the “reverse infection” protocol, different substrates were compared for infection efficiency: briefly, a ns-TiO₂ coated coverslip, a gelatin-coated coverslip and a plastic well were incubated with viral preparation (GFP-expressing virus) for 4 hours at 4-C (Virus-PEG correspond to a 10 fold concentrated viral preparation, Virus-supernatant correspond to the not concentrated viral preparation).

After a brief wash with PBS, melanocytes were plated on all the samples. After 72 hours cells were analysed with a fluorescence microscope. The infection efficiency and the mean fluorescence intensity were calculated for each sample using an image analysis software.

The results (FIG. 2B) show that the infections mediated by ns-TiO₂ in the absence of polybrene, are more efficient compared to others substrates (gelatin and plastic).

5. Retroviral Microarray with U2OS Cells

A slide was coated with an ns-TiO₂ film using supersonic cluster beam deposition. The slide was spotted with streptavidin, incubated and washed to eliminate the protein excess; subsequently, the biotinylated virus was spotted in the corresponding spots. Two different virus encoding fluorescent proteins locating at different cell-compartments—and staining the whole cell and the nucleolar dots, respectively—were used. This system allows the identification of the cell clusters specifically expressing the different viruses.

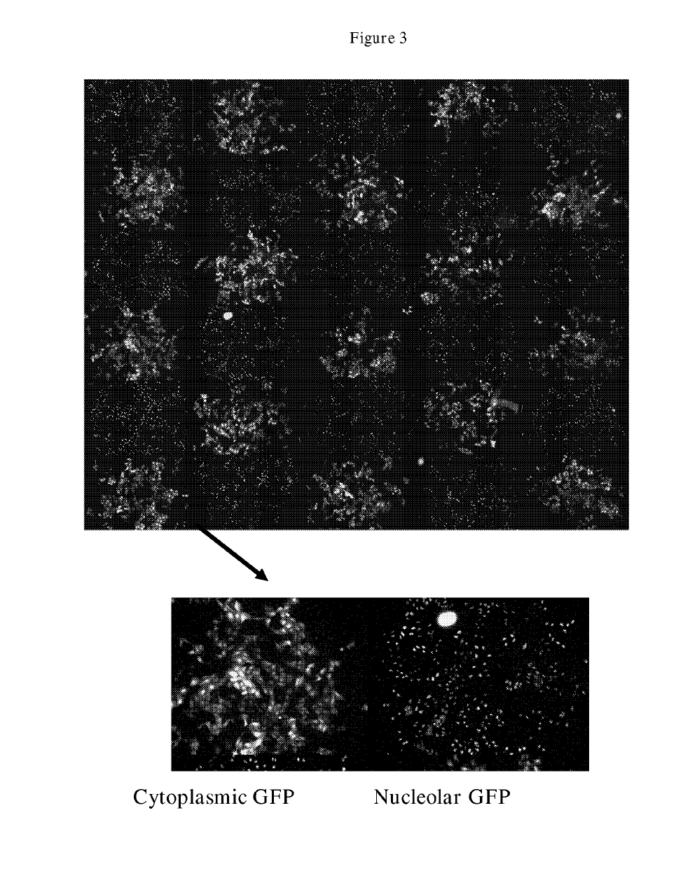

The slide was incubated to allow streptavidin/virus binding, washed and thereafter the cells were plated. After a period of 72 hours, the slide was fixed with 4% paraformaldehyde for 10 min and the cell nuclei were stained with DAPI. Image acquisition and analysis was carried out with an automated microscope. The results are illustrated in FIG. 3.

1.-10. (canceled)

11. A method for cell infection with viruses in vitro comprising the steps of:
(a) providing a solid support of a biocompatible substrate material being at least partially coated with a nanostructured TiO₂ film; and
(b) culturing cells on the nanostructured TiO₂ film-coated support in the presence of an infecting virus.

12. The method of claim 11 comprising the further steps of:
(c) contacting the nanostructured TiO₂ film-coated support with viruses prior to culturing cells in the support;
(d) contacting the virus adhered on the surface of the nanostructured TiO₂ film-coated support with a cell preparation; and
(e) culturing the cells for a time sufficient for the infection to occur.

13. A method for cell infection with viruses in vitro comprising the steps of:
(a) providing a solid support of a biocompatible substrate material being at least partially coated with a nanostructured TiO₂ film;
(b) immobilising streptavidin, avidin or neutravidin on the nanostructured TiO₂ film coating;
(c) contacting the nanostructured TiO₂ film-coated support with a biotinylated virus so as to form a complex of biotinylated viruses with the immobilised streptavidin, avidin or neutravidin;
(d) contacting the complex with a cell preparation; and
(e) culturing the cells for a time sufficient for the infection to occur.

14. A method for cell infection with viruses in vitro comprising the steps of:
(a) providing a solid support of a biocompatible substrate material being at least partially coated with a nanostructured TiO₂ film;
(b) adding a cell preparation to the nanostructured TiO₂ film-coated support;
(c) culturing the cells for an appropriate period of time;
(d) adding a viral supernatant;
(e) culturing the cells for a time sufficient for the infection to occur.

15. The method of claim 11 wherein the viruses are retroviruses, adenoviruses, adeno-associated viruses (AAV) and any other viruses that can be utilized as vectors for genetic manipulation of cells.

16. The method of claim 11 wherein the viruses are genetically modified.

17. (canceled)

18. Implantable particles or device of a nanostructured TiO₂ film-coated biocompatible material loaded with viruses.

19. A method for gene therapy ex vivo comprising the steps of:
(a) recovering cells to be genetically modified from a patient;
(b) establishing a primary cell culture from the recovered cells;
(c) infecting the cells by providing a solid support of a biocompatible substrate material being at least partially coated with a nanostructured TiO₂ film; and culturing cells on the nanostructured TiO₂ film-coated support with a virus that carries genetic information;
(d) and re-administering the infected cells to the patient.

20. A method for gene therapy in vivo comprising the steps of:
(a) providing particles or a device of a nanostructured TiO₂ film-coated biocompatible material;
(b) loading the particles or device with viruses; and
(c) implanting the virus-loaded particles or device into a tissue of a patient.

21. The method of claim 19 wherein the viruses are retroviruses, adenoviruses, adeno-associated viruses (AAV) and any other viruses that can be utilized as vectors for genetic manipulation of cells.

22. The method according to claim 19 wherein the viruses are genetically modified.

23. A method for cell replacement therapy comprising the steps of:
(a) providing particles or a device of a nanostructured TiO₂ film-coated biocompatible material;
(b) loading the particles or device with cells to be replaced in a patient; and
(c) implanting the cell-loaded particles or device into a tissue of a patient.

24. The method of claim 23 wherein the cells are genetically modified.

25. A solid support fabricated from a biocompatible substrate material which is at least partially coated with a nanostructured TiO₂ film having viruses and/or cells immobilised on the surface thereof.

26. The solid support of claim 25 wherein the film of nanostructured TiO₂ consists of TiO₂ nanoparticles with a diameter below 20 nm embedded in an amorphous TiO₂ matrix with a density of below 75% of bulk TiO₂ density.

27. The solid support of claim 25 which comprises a slide, dish, flask, plate, coverslip, fiber, foam, particle, membrane, porous scaffold, mesh or implant.

28. The solid support of claim 25 wherein the biocompatible substrate material is glass, plastic, ceramic, metal or a biodegradable or undegradable biopolymeric material.

29. The solid support of claim 25 wherein the viruses are retroviruses, adeno-viruses, adeno-associated viruses (AAV) and any other viruses that can be utilized as vectors for genetic manipulation of cells.

30. The solid support of claim 34 wherein the viruses are genetically modified.

31. The solid support according to claim 25 wherein biotinylated viruses are immobilised on the nanostructured TiO₂ film carrying streptavidin avidin or neutravidin.

32. The solid support according to claim 25 wherein the nanostructured TiO₂ film is in the form of a micro- or nano-pattern.

33. The solid support according to claim 32 wherein viruses carrying different genetic inserts are spotted on the surface of the nanostructured TiO₂ film.

34. A method for the production of a solid support, comprising the steps of:
fabricating the solid support from a biocompatible substrate material;
depositing a nanostructured TiO₂ film at least a portion of the substrate material by nanoparticle deposition from a gas-phase; and
contacting the surface of the nanostructured TiO₂ film with viruses and/or cells.

35. The method of claim 34 wherein the nanoparticle deposition from the gas-phase is carried out by means of supersonic cluster beam deposition (SCBD) using a pulsed microplasma cluster source.

36. A method of virus-mediated gene delivery to cells, comprising the steps of:
(a) providing a solid support of a biocompatible substrate material being at least partially coated with a nanostructured TiO₂ film;
(b) contacting the nanostructured TiO₂ film-coated support with gene delivering viruses;
(c) contacting the virus adhered on the surface of the nanostructured TiO₂ film-coated support with a cell preparation; and
(d) culturing the cells for a time sufficient for gene delivery to occur.
